(19) United States
(12) Patent Application Publication
(10) Pub. No.: US 2026/0327662 A1
(43) Pub. Date: Oct. 22, 2026

(54) A METHOD FOR PREPARING AN AQUEOUS SOLUTION CONTAINING GLYCEROL

(71) Applicant: ANNIKKI GMBH, Raaba-Grambach (AT)

(72) Inventors: Nicole STAUNIG, Raaba-Grambach (AT); Maria DUPONT, Raaba-Grambach (AT)

(21) Appl. No.: 19/122,696

(22) PCT Filed: Nov. 9, 2023
(86) PCT No.: PCT/EP2023/081325

§ 371 Date: Apr. 18, 2025

(30) Foreign Application Priority Data
Nov. 9, 2022 (EP) 22206345.5

(51) Int. Cl. C12P 7/56; C12N 9/04; C12N 9/88; C12P 7/20

(52) U.S. Cl. CPC C12P 7/56; C12N 9/0006; C12N 9/88; C12P 7/20; C12Y 101/01001; C12Y 401/02; C12Y 402/01

(57) ABSTRACT

A method for preparing an aqueous solution containing glycerol and a salt of pyruvic acid or lactic acid by treating a salt of gluconic acid and/or galactonic acid in an aqueous alcohol-containing solution in vitro with an enzyme system comprising a dehydratase, an aldolase, an alcohol dehydrogenase together with a cofactor, a glyceraldehyde reductase together with a cofactor and, optionally, a lactate dehydrogenase together with a cofactor, whereby an aqueous solution containing glycerol and a salt of pyruvic acid or lactic acid is obtained, from which the enzyme system is separated and which is optionally concentrated. (FIG. 1)

The invention relates to a method for preparing an aqueous solution containing glycerol and additionally a salt of pyruvic acid (pyruvate) or lactic acid (lactate).

Aqueous solutions containing glycerol and pyruvate or lactate are important starting materials for the production of various cosmetic products. This also applies to glycerol, pyruvate and lactate as such.

The reduction of pyruvate produces lactic acid or lactate, respectively, which has a wide range of applications. Lactic acid is also used, for example, as an acidifier in the food industry, as a descaling agent, pH regulator or cleaning agent in the chemical industry, as an additive in anti-acne creams or moisturisers in the cosmetics industry as well as a precursor for acrylic acid or ethyl lactate (Wee et al., 2006). In addition, lactic acid may also be used for skin peeling (chemical peeling) (Smith, 1996). Bifunctional lactic acid may also be polymerised to produce polylactide (PLA), a biodegradable and biocompatible plastic material with applications in the fields of the packaging, textile, electronics and medical industries (Balla et al., 2021).

In recent years, interest has shifted to methods that utilise highly selective conversions via enzyme cascade reactions to obtain target chemicals in one step (one-pot). EP2700714A1, U.S. Pat. No. 8,859,247B2 and EP2204453B1 describe a portfolio of cascade reactions, in which glucose is converted into two molecules of pyruvate in a cascade of five enzymatic reactions. Starting from glucose, the cascade follows the non-phosphorylating Entner-Doudoroff pathway to D-glyceraldehyde and pyruvate (sugar oxidation, dehydration and aldol cleavage). D-glyceraldehyde is then further oxidised to D-glycerate and subsequently dehydrated to pyruvate. This then serves as a platform for further transformations into a range of amino acids and alcohols. The enzymes of the cascade thereby comprise a glucose dehydrogenase, a promiscuous dihydroxy acid dehydratase (which catalyses dehydration of gluconate and D-glycerate), a 2-keto-3-deoxygluconate aldolase and an aldehyde dehydrogenase. By using also a promiscuous dehydrogenase, which accepts both glucose and D-glyceraldehyde as substrates, the system may be minimised to three enzymes.

The process described in EP2700714A1 for the formation of pyruvate from glucose was taken up by Sutiono et al. and extended to include glycerol as the starting material. The conversion of glucose to two molecules of pyruvate is realised in five steps (see previous paragraph). Glycerol must first be oxidised to D-glyceraldehyde, which is then converted to pyruvate in two steps. The main focus of this study was the characterisation of a promiscuous dehydratase from Paralcaligenes ureilyticus, which enables the dehydration of both gluconate and D-glycerate. Using an enzyme cascade containing the aforementioned dehydratase, 25 mM glucose (4.5 g/l) could be converted to approx. 45 mM ethanol (=90% conversion) within 12 h in the presence of 5 mM NAD+ (Sutiono et al., 2020).

Glycerol is obtained by reducing D-glyceraldehyde. Glycerol may be used to produce valuable downstream products such as 1,3-propanediol, which is used in the synthesis of polymers, in the production of cosmetics or in lubricants, or acrolein (2-propenal), a starting material for acrylic acid or its esters. Glycerol itself is used in cosmetics, food and pharmaceuticals, for example.

Glycerol and lactic acid also serve as starting materials for the synthesis of glycerol lactate esters (such as glycerol trilactate), which may be used as dietary supplements during physical exercises and subsequent regeneration and recovery, as lactic acid serves as an energy source for the heart and skeletal muscle (U.S. Pat. No. 6,743,821 B2).

As mentioned above, both glycerol and lactic acid (and in part also pyruvic acid) are used in cosmetic products, such as skin peels (Smith, 1996; Prestes et al., 2013; Ghersetich et al., 2004; O'Connor et al., 2018) or ointments (Pharmacy of the Heidelberg University Hospital, 2015). In order to manufacture such products, the individual pure substances are mixed together.

Glycerol may be produced by means of hydrolysis of triglycerides with alkaline lyes (saponification) or water (Tan et al., 2013; Rodrigues et al., 2017). Furthermore, fermentative methods for the production of glycerol (e.g. starting from glucose or sucrose) have also been known (e.g. Wang et al., 2001; Aslankoohi et al., 2015; U.S. Pat. No. 8,129,170 B1).

On a large scale, glycerol is mainly produced as a by-product of the biodiesel production, in which fats and oils are reacted with methanol, thus forming fatty acid methyl esters and glycerol (Tan et al., 2013; Rodrigues et al., 2017).

The use of methanol has a fundamental disadvantage: it is currently produced to a large extent from natural gas (approx. 65%) and crude oil (approx. 35%), i.e. from non-renewable carbon sources. Only around 0.2% comes from renewable resources (IRENA & METHANOL INSTITUTE, 2021). The current large-scale production of glycerol is therefore dependent on the consumption of non-renewable carbon sources.

Enzymes for the NADPH-dependent reduction of dihydroxyacetone and D- or L-glyceraldehyde are known from Liepins et al. (2006).

From the recently (2022) published paper by Wang et al. (Applied Microbiology and Biotechnology, 106(9-10)), it is known that L-lactate can be produced biosynthetically from glycerol and glucose. For the regeneration of NADH from NAD*, which is needed for the reduction of pyruvate, Wang et al. recommend a formate dehydrogenase.

In prior art, therefore, there is not known any method for preparing an aqueous solution containing glycerol and a salt of pyruvic acid (pyruvate) or lactic acid (lactate), which starts from renewable raw materials and leads to the desired products in a single biosynthesis step (one-pot biosynthesis).

This is precisely where the present invention comes in, thereby posing the task of providing a method for preparing an aqueous solution containing glycerol and a salt of pyruvic acid or lactic acid, as used, for example, in the cosmetics industry, which is based exclusively on a renewable carbon source and may additionally also be designated as ‘vegan’, which is in particular of importance when the aqueous solution is used in the cosmetics industry.

### DETAILED DESCRIPTION OF THE INVENTION

This problem is solved according to the invention by treating a salt of gluconic acid and/or galactonic acid in an aqueous, alcohol-containing solution in vitro with an enzyme system comprising a dehydratase, an aldolase, an alcohol dehydrogenase together with cofactor, a glyceraldehyde reductase together with cofactor and, optionally, a lactate dehydrogenase together with cofactor, whereby an aqueous solution containing glycerol and a salt of pyruvic acid or lactic acid is obtained, from which solution the enzyme system is separated and which is optionally concentrated.

In the method according to the invention, hence, the cofactor of the alcohol dehydrogenase is regenerated with an alcohol, in particular a C₁₋₄ alcohol, preferably isopropanol. Surprisingly, it has been shown that the regeneration of the cofactor with an alcohol is essential to solve the task of the invention. Regeneration with formate does not lead to the desired result (see comparative example below).

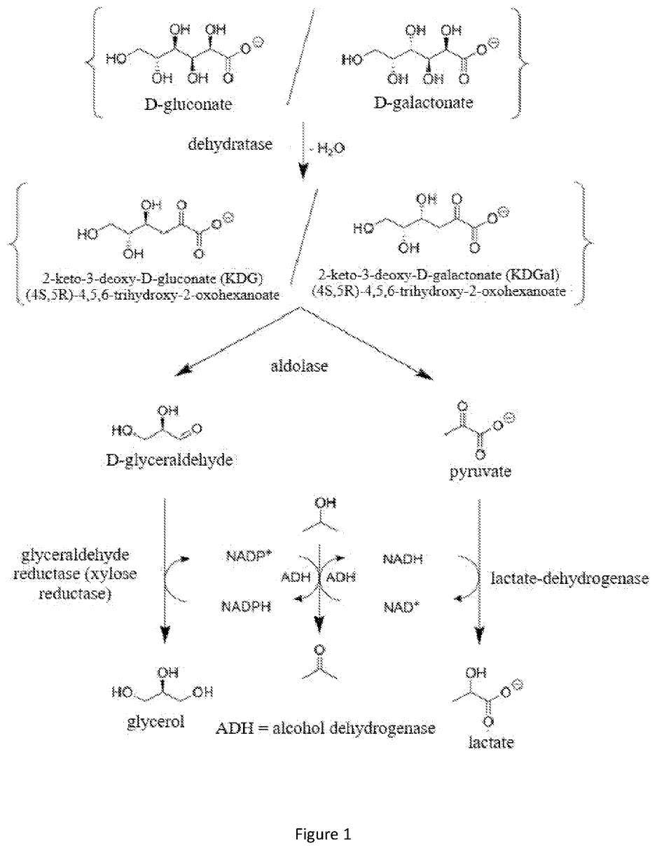

The method according to the invention is schematically shown in the attached FIG. 1. An alcohol dehydrogenase is used to regenerate the cofactor NADPH or NADH, although two different alcohol dehydrogenases may also be used.

In this patent application, the terms D-glucose is used interchangeably with glucose, D-gluconate interchangeably with gluconate, gluconate interchangeably with gluconic acid, D-galactonate interchangeably with galactonate, galactonate interchangeably with galactonic acid, D-glycerolaldehyde interchangeably with D-glyceraldehyde or glyceraldehyde, respectively, and lactic acid interchangeably with lactate.

Gluconic acid (or its anion gluconate) is a sugar acid derived from glucose, which is an important industrial chemical. Glucose in turn occurs in bound form (cellulose) in biomass and is the most common monosaccharide in nature. In general, straw, wood or waste products from the food industry, such as cereal chaff (e.g. oat husks) contain celluloses and hemicelluloses, from which monomer sugars may be released in varying compositions, primarily the hexoses glucose, mannose and galactose, the pentoses xylose and arabinose, as well as acids derived from the sugars (e.g. glucuronic acid). In maize husks, for example, 64% glucose, 4% galactose, 2% mannose, 10% arabinose and 16% xylose are found in relation to the total sugar (Hromádková & Ebringerová, 1995), whereas in wheat straw it is 56% glucose, 1% galactose, 2% mannose, 5% arabinose and 30% xylose (Collins et al., 2014). Methods for the efficient separation of the C₆ and Cₛ sugars allow these material stream types to be further treated separately (e.g. WO 2011/014894).

Gluconate is easily chemically accessible by oxidation at the C₁ position of glucose. The fermentative production of gluconate using Aspergillus niger or Gluconobacter oxydans is the most important technique. However, gluconate may also be obtained enzymatically from glucose using glucose oxidase, with atmospheric oxygen serving as the oxidising agent. The resulting hydrogen peroxide is harmful to the enzyme used and must therefore be removed, e.g. by using catalase (Kornecki et al., 2020).

In organisms, gluconate is an important intermediate in the so-called Entner-Doudoroff pathway (ED pathway), in which two molecules of pyruvate and one molecule of adenosine triphosphate (ATP) are produced from glucose. The precise configuration of the ED pathway depends on the microorganism; for example, it will occur in bacteria such as Escherichia coli. For archaea, further variations such as the non-phosphorylating, semi-phosphorylating or branched ED pathway are known. The core step in all pathways is the aldol cleavage of a C₆ sugar acid (2-keto-3-deoxygluconate or 2-keto-3-deoxy-6-phosphogluconate, depending on the microorganism) into two C₃ products (pyruvate and glycerolaldehyde or glycerolaldehyde-3-phosphate) (Entner & Doudoroff, 1952; Conway, 1992; Peekhaus & Conway, 1998; Reher et al., 2010; Sutter et al., 2016).

In the ED pathway, the intermediate D-glyceraldehyde is exclusively oxidised to D-glyceric acid, which is then further converted to pyruvate via several steps. Pyruvate is a central intermediate in cellular metabolism and, as such, an important starting material for the production of alcohols, amino acids, or 2,3-butanediol (Sutiono et al., 2020), making the ED pathway interesting for biosynthetic applications. Moreover, pyruvate may also be used for the treatment of sun-damaged facial skin in the form of peelings (Ghersetich et al., 2004).

Due to the promiscuity of the enzymes in the Entner-Doudoroff pathway, not only glucose but also galactose (the C₄ epimer) may be converted to pyruvate and D-glyceraldehyde by the same enzymes. This has been well proven for the hyperthermophilic archaeon Sulfolobus solfataricus (Lamble et al., 2003; Theodossis et al., 2004).

The solutions prepared according to the invention may serve as a basis for the production of various cosmetic products and are entirely based on renewable raw materials (C₆ sugar acids, for example, obtained from monosaccharides present in biomass) and are furthermore vegan. Thus, the inventive method circumvents the use of methanol from fossil sources for the production of glycerol.

It has furthermore surprisingly been shown that the objective of the present invention may be solved when the conversion is carried out in vitro, meaning that the enzymes are present as such in the aqueous solution. There need not be carried out any fermentative processes. Another advantage of the method according to the invention is that the conversion may also be conducted under non-physiological conditions, such as high substrate concentrations, which are concentrations, at which a fermentative method could not operate at all.

The term “enzyme system” refers to the totality of the enzymes used, namely the dehydratase, the aldolase, the glyceraldehyde reductase together with cofactor, the lactate dehydrogenase together with cofactor, as well as an alcohol dehydrogenase together cofactor, such as a NAD-specific alcohol dehydrogenase and/or a NADP-specific alcohol dehydrogenase. These enzymes together with cofactors may be added to the aqueous solution as such (enzyme isolates). However, it is also possible to suspend cells that express the specified enzymes in the aqueous solution. Depending on the reaction conditions, the cells may remain intact or burst, releasing the enzyme into the aqueous environment. In the presence of intact cells, the substrates diffuse into the cell, where they are converted by the enzyme, and the products then subsequently diffuse back out. In this case, the term “enzyme system” is understood to refer to a cell suspension. Further, the term “enzyme system” is to be understood as a homogenate that may be obtained from the cell suspension, for example, by exposing the cells to ultrasound to cause them to burst. Finally, the term “enzyme system” is also to be understood as lysates that are obtained from the homogenates by separating the solid cellular components.

According to a preferred embodiment of the method according to the present invention, there is used an alkali salt, in particular the sodium or potassium salt of gluconic acid and galactonic acid.

In further preferred embodiments of the method according to the present invention, the concentration of the alkali salt of gluconic acid and/or galactonic acid in the aqueous solution is between 50 and 350 g/I, 150 and 300 g/I, and more preferably between 150 and 250 g/I.

Additionally, it is preferred that the method according to the invention is carried out at a temperature between 20 and 50 °C, between 25 and 40 °C, and even more preferably between 30 and 40 °C.

The particularly preferred pH range for the reaction is between 7.5 and 8.5.

In another preferred variant of the method according to the present invention, the enzymes forming the enzyme system are present in a suspension, in the homogenate, and/or in the lysate of the corresponding cells forming them. It has been shown that when using suspensions, lysates, and/or homogenates, the cofactors of the dehydrogenases and reductases do not need to be added separately, as they are already present in the suspension, lysate, and/or homogenate.

In this context, suspension refers to a suspension of resting cells. These are harvested after cultivation (separated from the nutrient medium) and suspended in a suitable buffer system. In contrast to fermentative methods, which also work with whole cells, the resting cells can no longer grow due to the removal of carbon sources and nutrients, but rather solely serve for the conversion of substrates (Lin & Tao, 2017). Homogenate in this context refers to a physically and/or chemically treated suspension (e.g., treated by means of pressure, lysozyme, or ultrasound), wherein the cellular components are released from the cells. A lysate is obtained when the insoluble cellular components of the homogenate are removed, for example, through filtration or centrifugation (see Production of the Enzymes for details).

In a further variant, the enzymes may also be present in powdered form, immobilised in or on a solid support material, or part of a fusion protein.

In order to enable the conversion of both C₆-sugar acids, natural enzyme promiscuity is used, which allows one enzyme to catalyse up to two target reactions in order to minimise the enzyme cascade.

According to another preferred aspect of the invention, all reaction steps of the method, as well as the regeneration of the cofactors, take place in a single reaction vessel (one-pot reaction), thus avoiding the costly isolation of intermediates. In the preferred embodiment, all enzymes are used at the beginning of the reaction.

The conversion of gluconate via (4S,5R)-4,5,6-trihydroxy-2-oxohexanoate (2-keto-3-deoxygluconate=KDG) or galactonate via (4R,5R)-4,5,6-trihydroxy-2-oxohexanoate (2-keto-3-deoxygalactonate=KDGal) to pyruvate and D-glyceraldehyde thus comprises a dehydratase and an aldolase.

The dehydratase used in the method may be derived from one of the following groups: EC 4.2.1.5 (arabonate dehydratase), 4.2.1.6 (galactonate dehydratase), 4.2.1.7 (altronate dehydratase), 4.2.1.8 (mannonate dehydratase), 4.2.1.9 (dihydroxy acid dehydratase), 4.2.1.25 (L-arabonate dehydratase), 4.2.1.39 (gluconate dehydratase), 4.2.1.40 (glucuronate dehydratase), 4.2.1.42 (galactarate dehydratase), 4.2.1.67 (D-fuconate dehydratase), 4.2.1.68 (L-fuconate dehydratase), 4.2.1.82 (xylonate dehydratase), 4.2.1.140 (gluconate/galactonate dehydratase), 4.2.1.146 (L-galactonate dehydratase), 4.2.1.156 (L-talarate dehydratase), 4.2.1.158 (galactarate dehydratase, D-threo-forming), and 4.2.1.176 (L-lyxonate dehydratase), wherein the group 4.2.1.25 (L-arabonate dehydratase) is particularly preferred.

The aldolase used in the method may be derived from one of the following groups: EC 4.1.2.18 (2-dehydro-3-deoxy-L-pentonate aldolase), 4.1.2.20 (2-dehydro-3-deoxyglucuronate aldolase), 4.1.2.21 (2-dehydro-3-deoxy-6-phosphogalactonate aldolase), 4.1.2.28 (2-dehydro-3-deoxy-D-pentonate aldolase), 4.1.2.29 (5-dehydro-2-deoxyphosphogluconate aldolase), 4.1.2.51 (2-dehydro-3-deoxy-D-gluconate aldolase), 4.1.2.52 (4-hydroxy-2-oxoheptanedioate aldolase), 4.1.2.53 (2-keto-3-deoxy-L-rhamnonate aldolase), 4.1.2.54 (L-threo-3-deoxy-hexulose aldolase), 4.1.2.55 (2-dehydro-3-deoxy-phosphogluconate/2-dehydro-3-deoxy-6-phosphogalactonate aldolase), and 4.1.3.39 (4-hydroxy-2-oxovalerate aldolase), wherein the group 4.1.2.55 (2-dehydro-3-deoxy-phosphogluconate/2-dehydro-3-deoxy-6-phosphogalactonate aldolase) is particularly preferred.

The reductase used for the reduction of D-glyceraldehyde (glyceraldehyde reductase) is preferably derived from the group of D-xylose reductases (EC 1.1.1.307).

The dehydrogenase used for the reduction of pyruvate to lactate is derived from the group EC 1.1.1.27 (L-lactate dehydrogenase).

The alcohol dehydrogenases used for the regeneration of the cofactors are preferably from the groups EC 1.1.1.1 (NAD-dependent ADH) or EC 1.1.1.2 (NADP-dependent ADH).

The enzymatic strategy presented herein minimises the number of enzymes, thus enabling a highly efficient and cost-effective/price-competitive bioproduction process.

Another aspect of the present invention relates to an aqueous solution comprising glycerol and a salt of pyruvic acid or lactic acid, obtainable by a method according to the invention.

Using the following examples, preferred embodiments of the invention will further be described.

### Materials

Sodium gluconate, lithium salt of 2-keto-3-deoxygluconic acid, D-glyceraldehyde, sodium pyruvate, sodium L-lactate, zinc chloride, IPTG (isopropyl-β-D-thiogalactopyranoside), and HEPES (2-(4-(2-hydroxyethyl)-1-piperazinyl)ethanesulfonic acid) were obtained from Sigma-Aldrich, glycerol, magnesium chloride hexahydrate, potassium dihydrogen phosphate, dipotassium hydrogen phosphate, NAD⁺ disodium salt, NADH disodium salt, NADP₊ disodium salt, and sodium dodecyl sulfate (SDS) were obtained from Carl Roth, NADPH tetranatrium salt, lysozyme, and methanol were acquired from PanReac AppliChem (ITW Reagents), and triethanolamine was obtained from Chem-Lab NV. Potassium D-galactonate was produced according to the literature method (Moore & Link, 1940) from galactose.

### Production of the Enzymes

For the recombinant enzyme production in an Escherichia coli strain, the gene to be expressed was first amplified in a PCR using genomic DNA or its synthetic equivalent adapted to the codon usage of E. coli as a template, along with specific oligonucleotides that additionally contain recognition sequences for restriction endonucleases, and it was then isolated from the reaction mixture. Following the digestion of the nucleic acids with the restriction enzymes SphI and HindIII, the gene fragment coding for the target enzyme was ligated into the SphI and HindIII-cut backbone of the expression vector pQE70-Kan. The ligation product was transformed into chemically competent E. coli cells Top10F, and the resulting colonies were used for plasmid isolation and restriction analysis.

The result of the cloning step was verified by means of restriction enzyme digestion and DNA sequencing. The resulting construct carries the target gene under the IPTG-inducible T5 promoter.

For the overexpression of the enzyme in E. coli, the resulting expression plasmid was transformed into the competent expression cells RB791. After 24 hours of incubation at 37 °C, the resulting colonies were inoculated into LB medium for expression tests.

The following day, expression cultures were inoculated at an optical density (OD₅₅₀) of 0.02 and shaken at 37 °C until an OD₅₅₀ of 0.3 was reached. Subsequently, the temperature was lowered to 25 °C, and the cultures were induced with 0.1 mM IPTG upon reaching an OD₅₅₀ of 0.5. After 22 hours, the cultures were harvested (separated from the medium by centrifugation into a cell pellet) and analysed for the expression of the recombinant enzyme using SDS gel electrophoresis and an activity assay (utilised in a use-test or optical-enzymatic assay).

### Preparation of Cell Suspensions

In order to prepare cell suspensions, the cell pellets produced according to the method above were weighed into a suitable vessel and a buffer was added (e.g., triethanolamine (TEA)-HCl buffer) while stirring in an ice bath. The biomass mass fraction typically amounts to 20%, with the remainder consisting of the buffer.

### Preparation of Homogenates Via Sonication

Lysozyme was added to the cell suspension prepared above at a concentration of 0.5 mg/ml. A Branson Sonifier 450 was used for cell disruption. The suspension was transferred into a 5 ml Eppendorf vial. The metal tip of the apparatus was then immersed in the suspension, after which the suspension was subjected to three cycles of 15 ultrasonic pulses each (device settings: Timer=15; Duty Cycle=50; Output Control=3-5). In this way, the homogenate was obtained as a mixture of disrupted cells and buffer.

### Preparation of Lysates by Centrifugation

The homogenate was centrifuged for 10 minutes at 4 °C and 16,000 rpm (Eppendorf Centrifuge 5417R) to separate the insoluble cell fragments and obtain the lysate.

TABLE 1
Enzyme types and donor organisms for the enzymes used.

| enzyme type (EC class) | catalysed reaction(s) | donor organisms | literature |
|---|---|---|---|
| dehydratase | gluconate → KDG; | Azotobacter vinelandii | (Setubal et al., 2009); |
| (EC 4.2.1.25) | galactonate → KDGal | DJ | (NCBI Protein |
| | | | Database: |
| | | | ACO81022.1) |
| aldolase | KDG / KDGal → | Sulfolobus | Wolterink-van Loo et |
| (EC 4.1.2.55) | pyruvate + | acidocaldarius | al., 2009) |
| | D-glyceraldehyde | | |
| D-xylose reductase | D-glyceraldehyde → | Debaryomyces | (de Faria et al., 2009) |
| (EC 1.1.1.307) | glycerol | hansenii | |
| L-lactate dehydrogenase | pyruvate → | Oryctolagus cuniculi | (Zheng et al., 2004) |
| (EC 1.1.1.27) | L-lactate | (rabbit muscle) | |
| alcohol dehydrogenase | isopropanol → acetone | Thermoanaerobium | (Lamed & Zeikus, |
| (NADP) | | brockii | 1980) |
| (EC 1.1.1.2) | | | |
| alcohol dehydrogenase | isopropanol → acetone | Acetobacter | (Chinnawirotpisan et |
| (NAD) | | pasteurianus | al., 2003) |
| (EC 1.1.1.1) | | | |

### Analytical Methods

### High Performance Anion Exchange Chromatography

Substrate turnover and product concentrations were determined using HPAEC (High Performance Anion Exchange Chromatography). A Dionex ICS6000 system equipped with an AS-AP autosampler was employed for this purpose. The measurement of the organic acids and their anions (gluconate, KDG, KDGal, pyruvate, and lactate) was carried out by means of conductivity detection (CD) coupled with a Dionex AERS 500 electrolytically regenerated suppressor in external water mode. A Dionex IonPac AS11-HC-4 μm column, with an appropriate precolumn and an NaOH gradient, was utilised for the separation of the analytes. The mobile phase was additionally pre-treated with a Dionex ATC Anion Trap Column.

### High Performance Liquid Chromatography

In order to quantify D-glyceraldehyde and glycerol via HPLC (High Performance Liquid Chromatography), an Agilent HPLC 1260 Infinity II Series system was used. Detection was carried out using a refractive index detector (RI detection). A Phenomenex Rezex ROA-Organic Acid H+ (8%) column, together with a corresponding precolumn, was used for the measurement, and elution was performed isocratically with 1 mM sulphuric acid.

### Determination of Enzyme Activities (Optical-Enzymatic Assay)

Enzyme activities in the homogenates or lysates, respectively, were determined using a Shimadzu UV-1900 spectrophotometer. The formation or consumption, respectively, of NADH was monitored at a wavelength of 340 nm by tracking changes in the absorption. Measurements were conducted with 0.2 mM cofactor (NAD⁺ or NADH). For this purpose, 20 μl of a 10 mM stock solution of the cofactor was placed in a cuvette (Greiner bio-one Semi-Micro Cuvette made of polystyrene), and the desired pH was adjusted using 100 mM TEA-HCl buffer (870 μl). Following the temperature equilibration of the cuvette, 10 μl of homogenate or lysate (diluted or undiluted), respectively, and 100 μl of substrate solution were added, and measurement was started immediately thereafter. Measurements were routinely performed at 25 °C. The enzyme activity of the may be determined in U/ml (based on the volume of the lysate) or U/g (based on the biomass used for production) using the extinction coefficient of NADH at 340 nm (ε=6220 L mol⁻¹ cm⁻¹). 1 U corresponds to 1 μmol of substrate turnover per minute (1 U=1 μmol/min=1.67×10⁻⁸ kat).

### General Information on the Processing of the Reaction Solution

The aqueous solution containing glycerol and lactate or glycerol and pyruvate, respectively, prepared according to the invention, is obtained by removing the cellular components through denaturation (e.g., via heat treatment) and subsequent filtration or centrifugation, respectively, after the completion of the reaction. In order to remove smaller cellular components, membrane filtration may additionally be performed. The thus obtained filtrate may then be concentrated to the desired concentration (e.g., 250 g/l glycerol or lactate) by evaporating the water.

The following examples describe preferred variants of the method according to the invention in greater detail. The suspensions, homogenates, and lysates used in these examples were prepared according to the methods described above.

### Example 1

### Conversion of Sodium Gluconate to Pyruvate and Glycerol

In a 2 ml glass vial, the following components were prepared: 25 μl of a 2000 mM TEA-HCl buffer (pH 8.5), 143 μl of deionised water, 80 μl of dehydratase lysate, 80 μl of aldolase lysate, 2 U of D-xylose reductase lysate, and 2 U of alcohol dehydrogenase lysate (NADP-specific). The reaction was started by the addition of 100 μl of a sodium gluconate solution (2296 mM). For the cofactor regeneration, 50 μl of isopropanol was added. The batch was incubated for a total of 48 hours at 30 °C with continuous shaking (1200 rpm, Eppendorf Thermomixer). 26 hours after the start of the reaction, 100 μl of an isopropanol/water mixture (3/7 v/v) was added to the batch. The total volume of the batch at the start of the reaction was 500 μl.

For processing, 20 μl of the batch was combined with 180 μl of ultrapure water and 200 μl of MeOH and incubated at 60 °C for 20 minutes (1200 rpm, Eppendorf Thermomixer). Following denaturation, the sample was centrifuged for 10 minutes at maximum g-force. The clear supernatant was diluted 1:250 and analysed using HPAEC (conductivity detection). For the HPLC, 150 μl of the clear supernatant was transferred to HPLC vials for analysis (RI detection). The results are presented in the following table.

| | | | conversion |
|---|---|---|---|
| | | concentration | after 48 h |
| | | [mm] | [%] |
| substrate | sodium | 459 | 64 |
| (t = 0 h) | gluconate | | |
| products | glycerol | 120 | 26 |
| (t = 48 h) | pyruvate | 39 | 8 |

In addition to the products listed in the table, the intermediates KDG and KDGal are also detected.

The table above indicates that gluconate is converted by the enzyme system into glycerol and pyruvate.

### Example 2

### Conversion of Sodium Gluconate to Lactate and Glycerol

In a 2 ml glass vial (vial 1), the following components were prepared: 25 μl of a 2000 mM TEA-HCl buffer (pH 8.5), 172 μl of deionised water, 80 μl of dehydratase lysate, 80 μl of aldolase lysate, 2 U of D-xylose reductase lysate, 2 U of alcohol dehydrogenase lysate (NADP-specific), 2 U of alcohol dehydrogenase lysate (NAD-specific), and 2 U of lactate dehydrogenase lysate. In another 2 ml glass vial (vial 2), a similar mixture was prepared, but with 122 μl (vial 2) of deionised water.

The reactions were started by the addition of 50 μl of a sodium gluconate solution (2296 mM) to vial 1 (vial 2: 100 μl). For the cofactor regeneration, 50 μl of isopropanol was added to each vial. The batches were incubated for a total of 48 hours at 30 °C with continuous shaking (1200 rpm, Eppendorf Thermomixer). 26 hours after the start of the reaction, 100 μl of an isopropanol/water mixture (3/7 v/v) was added to the batches. The total volume of the batches at the start of the reaction was 500 μl each.

For processing, x μl of the batch was combined with y μl of deionised water (x=40, y=160 for vial 1; x=20, y=180 for vial 2) and 200 μl of MeOH, and incubated at 60 °C for 20 minutes (1200 rpm, Eppendorf Thermomixer). Following denaturation, the samples were centrifuged for 10 minutes at maximum g-force. The clear supernatant was diluted 1:250 and analysed using HPAEC (conductivity detection). For the HPLC, 150 μl of the clear supernatant was transferred to HPLC vials for analysis (RI detection). The results are presented in the following table.

| | | | | conversion |
|---|---|---|---|---|
| | | | concentration | after 48 h |
| | | | [mm] | [%] |
| vial 1 - 230 mm substrate | | | | |
| | substrate | sodium | 230 | 58 |
| | (t = 0 h) | gluconate | | |
| | products | glycerol | 131 | 57 |
| | (t = 48 h) | lactate | 137 | 60 |
| vial 2 - 459 mm substrate | | | | |
| | substrate | sodium | 459 | 49 |
| | (t = 0 h) | gluconate | | |
| | products | glycerol | 191 | 42 |
| | (t = 48 h) | lactate | 190 | 41 |

In addition to the products listed in the table, the intermediates KDG and KDGal are also detected.

The tables above indicate that the method is also suitable for the reaction of a more concentrated substrate solution (459 mM).

### Example 3

### Influence of Various Enzyme Formulations (Suspension, Homogenate or Lysate) on the Conversion of Sodium Gluconate to Lactate and Glycerol

In a 2 ml glass vial, the following components were prepared: 25 μl of a 2000 mM TEA-HCl buffer (pH 8.5), 130 μl of deionised water, 80 μl of the dehydratase formulation (see the following tables), 80 μl of the aldolase formulation, 2 U of D-xylose reductase formulation, 2 U of NADP-specific alcohol dehydrogenase formulation, 2 U of NAD-specific alcohol dehydrogenase formulation, and 2 U of lactate dehydrogenase lysate. The reactions were started by the addition of 100 μl of a sodium gluconate solution (1145 mM). For the cofactor regeneration, 50 μl of isopropanol was added. The batches were incubated for a total of 48 hours at 30 °C with continuous shaking (1200 rpm, Eppendorf Thermomixer). 26 hours after the start of the reaction, 100 μl of an isopropanol/water mixture (3/7 v/v) was added to each reaction batch. The total volume of the batches at the start of the reaction was 500 μl each.

For processing, 40 μl of the batch was combined with 160 μl of ultrapure water and 200 μl of MeOH and incubated at 60 °C for 20 minutes (1200 rpm, Eppendorf Thermomixer). Following denaturation, the samples were centrifuged for 10 minutes at maximum g-force. The clear supernatant was diluted 1:250 and analysed using HPAEC (conductivity detection). For the HPLC, 150 μl of the clear supernatant was transferred into HPLC vials for analysis (RI detection). The results are presented in the following table.

| | | | conversion |
|---|---|---|---|
| | | concentration | after 48 h |
| | | [mm] | [%] |
| aldolase as suspension; L-lacDH | | | |
| as lysate;remaining enzymes as | | | |
| homogenate | | | |
| substrate | sodium | 229 | 54 |
| (t = 0 h) | gluconate | | |
| products | glycerol | 92 | 40 |
| (t = 48 h) | lactate | 80 | 35 |
| aldolase as suspension; | | | |
| remaining enzymes as lysate | | | |
| substrate | sodium | 229 | 58 |
| (t = 0 h) | gluconate | | |
| products | glycerol | 90 | 39 |
| (t = 48 h) | lactate | 76 | 33 |
| L-lacDH as lysate; remaining | | | |
| enzymes as homogenate | | | |
| substrates | sodium | 229 | 55 |
| (t = 0 h) | gluconate | | |
| products | glycerol | 102 | 45 |
| (t = 48 h) | lactate | 80 | 35 |
| all enzymes as lysate | | | |
| substrate | sodium | 229 | 55 |
| (t = 0 h) | gluconate | | |
| products | glycerol | 107 | 47 |
| (t = 48 h) | lactate | 98 | 43 |

In addition to the products listed in the table, the intermediates KDG and KDGal, as well as pyruvate, are also detected.

The tables above indicate that different enzyme formulations (suspensions, homogenates, and lysates) may be used for the enzyme system. The highest conversion rate is obtained when all enzymes are added in the form of lysates.

### Example 4

Influence of Cofactor Addition (NAD⁺ and NADP⁺) on the Conversion of Sodium Gluconate to Lactate and Glycerol

In a 2 ml glass vial (vial 1), the following components were prepared: 25 μl of a 2000 mM TEA-HCl buffer (pH 8.5), 130 μl of deionised water, 80 μl of dehydratase lysate, 80 μl of aldolase lysate, 2 U of D-xylose reductase lysate, 2 U of NADP-specific alcohol dehydrogenase lysate, 2 U of NAD-specific alcohol dehydrogenase lysate, and 2 U of lactate dehydrogenase lysate. In a second 2 ml glass vial, a similar mixture was prepared, however, with 10 μl of a 10 mM NAD⁺ solution, 10 μl of a 10 mM NADP⁺ solution, and 110 μl instead of 130 μl of deionised water.

The reactions were started by the addition of 100 μl of a sodium gluconate solution (1145 mM) to both glass vials. For the cofactor regeneration, 50 μl of isopropanol was added. The batches were incubated for a total of 48 hours at 30 °C with continuous shaking (1200 rpm, Eppendorf Thermomixer). 26 hours after the start of the reaction, 100 μl of an isopropanol/water mixture (3/7 v/v) was added to each batch. The total volume of the batches at the start of the reaction was 500 μl each.

For processing, 40 μl of the batch was combined with 160 μl of ultrapure water and 200 μl of MeOH, and incubated at 60 °C for 20 minutes (1200 rpm, Eppendorf Thermomixer). Following denaturation, the samples were centrifuged for 10 minutes at maximum g-force. The clear supernatant was diluted 1:250 and analysed using HPAEC (conductivity detection). For the HPLC, 150 μl of the clear supernatant was transferred into HPLC vials for analysis (RI detection). The results are presented in the following table.

| | | | | conversion |
|---|---|---|---|---|
| | | | concentration | after 48 h |
| | | | [mm] | [%] |
| vial 1 - no addition of cofactor | | | | |
| | substrate | sodium | 229 | 55 |
| | (t = 0 h) | gluconate | | |
| | products | glycerol | 107 | 47 |
| | (t = 48 h) | lactate | 98 | 43 |
| vial 2 - addition of cofactor | | | | |
| (0.2 mM NAD⁺ and 0.2 mM | | | | |
| NADP⁺ final) | | | | |
| | substrate | sodium | 229 | 53 |
| | (t = 0 h) | gluconate | | |
| | products | glycerol | 103 | 45 |
| | (t = 48 h) | lactate | 100 | 43 |

In addition to the products listed in the table, the intermediates KDG and KDGal, as well as pyruvate, are also detected.

The tables above indicate that the addition of cofactor (0.2 mM NAD⁺ and 0.2 mM NADP*) at a sodium gluconate concentration of 229 mM does not entail any advantages regarding the conversion, as there is already present sufficient cofactor from the lysates used. However, at higher substrate concentrations, the addition of a cofactor may be necessary.

### Example 5

### Conversion of Potassium Galactonate to Pyruvate and Glycerol

In a 2 ml glass vial, the following components were prepared: 25 μl of a 2000 mM TEA-HCl buffer (pH 8.5), 147 μl of deionised water, 80 μl of dehydratase lysate, 80 μl of aldolase lysate, 2 U of D-xylose reductase lysate, and 2 U of alcohol dehydrogenase lysate (NADP-specific). The reaction was started by the addition of 100 μl of a potassium galactonate solution (1282 mM). For the cofactor regeneration, 50 μl of isopropanol was added. The batch was incubated for a total of 48 hours at 30 °C with continuous shaking (1200 rpm, Eppendorf Thermomixer). 26 hours after the start of the reaction, 100 μl of an isopropanol/water mixture (3/7 v/v) was added to the sample. The total volume of the batch at the start of the reaction was 500 μl.

For processing, 40 μl of the batch was combined with 160 μl of ultrapure water and 200 μl of MeOH, and incubated at 60 °C for 20 minutes (1200 rpm, Eppendorf Thermomixer). Following denaturation, the sample was centrifuged for 10 minutes at maximum g-force. The clear supernatant was diluted 1:250 and analysed using HPAEC (conductivity detection). For the HPLC, 150 μl of the clear supernatant was transferred into HPLC vials for analysis (RI detection). The results are presented in the following table.

| | | | conversion |
|---|---|---|---|
| | | concentration | after 48 h |
| | | [mm] | [%] |
| substrate | potassium | 256 | 72 |
| (t = 0 h) | galactonate | | |
| products | glycerol | 53 | 21 |
| (t = 48 h) | pyruvate | 24 | 10 |

In addition to the products listed in the table, the intermediates KDG and KDGal are also detected.

The table above indicates that the enzyme system also converts galactonate into glycerol and pyruvate.

### Example 6

### Conversion of Potassium Galactonate to Lactate and Glycerol

In a 2 ml glass vial, the following components were prepared: 25 μl of a 2000 mM TEA-HCl buffer (pH 8.5), 130 μl of deionised water, 80 μl of dehydratase lysate, 80 μl of aldolase lysate, 2 U of D-xylose reductase lysate, 2 U of NADP-specific alcohol dehydrogenase lysate, 2 U of NAD-specific alcohol dehydrogenase lysate, and 2 U of lactate dehydrogenase lysate. The reaction was started by the addition of 100 μl of a potassium galactonate solution (1282 mM). For the cofactor regeneration, 50 μl of isopropanol was added. The batch was incubated for a total of 48 hours at 30 °C with continuous shaking (1200 rpm, Eppendorf Thermomixer). 26 hours after the start of the reaction, 100 μl of an isopropanol/water mixture (3/7 v/v) was added to the sample. The total volume of the batch at the start of the reaction was 500 μl.

For processing, 40 μl of the batch was combined with 160 μl of ultrapure water and 200 μl of MeOH, and incubated at 60 °C for 20 minutes (1200 rpm, Eppendorf Thermomixer). Following denaturation, the sample was centrifuged for 10 minutes at maximum g-force. The clear supernatant was diluted 1:250 and analysed using HPAEC (conductivity detection). For the HPLC, 150 μl of the clear supernatant was transferred into HPLC vials for analysis (RI detection). The results are presented in the following table.

| | | | conversion |
|---|---|---|---|
| | | concentration | after 48 h |
| | | [mm] | [%] |
| substrate | potassium | 256 | 70 |
| (t = 0 h) | galactonate | | |
| products | glycerol | 84 | 33 |
| (t = 48 h) | lactate | 57 | 22 |

In addition to the products listed in the table, the intermediates KDG and KDGal, as well as pyruvate, are also detected.

The table above indicates that the enzyme system also converts galactonate into glycerol and lactate.

### Comparative Example

### Conversion of a Mixture of D-Glyceraldehyde and Pyruvate to Glycerol and Lactate

In two 2 ml Eppendorf vials, the following components were prepared: 100 μl of a 500 mM TEA-HCl buffer (pH 8.5; final concentration 100 mM), 0.1 U of lactate dehydrogenase lysate, 0.1 U of D-xylose reductase lysate, 10 μl of a 10 mM NAD⁺ solution (final concentration 0.2 mM), and 10 μl of a 10 mM NADP⁺ solution (final concentration 0.2 mM).

To vial 1, 0.5 μl of NAD-specific alcohol dehydrogenase lysate, 0.5 μl of NADP-specific alcohol dehydrogenase lysate, and 38.5 μl of a 10 vol % isopropanol solution (corresponding to a final concentration of 100 mM) were added. To vial 2, 0.5 μl of NAD-specific formate dehydrogenase lysate (from Starkeya novella, NCBI Protein Database: WP_013168047.1), 0.5 μl of NADP-specific formate dehydrogenase lysate (NADP-specific due to cofactor engineering; from Starkeya novella), and 100 μl of a 500 mM sodium formate solution (final concentration 100 mM) were added.

The reactions were started by the addition of 100 μl of a substrate solution (50 mM sodium pyruvate and 50 mM D-glyceraldehyde in deionised water). The batches were incubated for 1 hour at 30 °C and 1200 rpm in an Eppendorf Thermomixer. The total volume of each batch at the start of the reaction was 500 μl (filled with deionised water).

For processing, 200 μl of a batch was combined with 200 μl of MeOH and incubated at 60 °C for 20 minutes (1200 rpm, Eppendorf Thermomixer). Following denaturation, the samples were centrifuged for 10 minutes at maximum g-force. In order to quantify the analytes, 150 μl of the clear supernatant was transferred into HPLC vials and analysed by means of HPLC (RI detection).

In vial 1 (cofactor regeneration via alcohol dehydrogenases and isopropanol), 79% of the pyruvate (starting concentration 10 mM) was converted to lactate, and 61% of the D-glyceraldehyde (starting concentration 10 mM) was reduced to glycerol. In vial 2 (cofactor regeneration via formate dehydrogenases and sodium formate), only 32% of the pyruvate was converted to lactate, and glycerol could not be detected at all.

The results show that cofactor regeneration via the alcohol dehydrogenases, using the same biomass (0.5 μl of 20% lysate), leads to higher conversions compared to regeneration with the formate dehydrogenases.

### LITERATURE

Wee, Y.-J., Kim, J.-N., & Ryu, H.-W. (2006). Biotechnological Production of Lactic Acid and Its Recent Applications. Food Technology & Biotechnology, 44(2), 163-172. https://www.ftb.com.hr/80-volume-44-issue-no-2/445-biotechnological-production-of-lactic-acid-and-its-recent-applications
Smith, W. P. (1996). Epidermal and dermal effects of topical lactic acid. Journal of the American Academy of Dermatology, 35(3), 388-391. https://doi.org/10.1016/s0190-9622(96)90602-7
Balla, E., Daniilidis, V., Karlioti, G., Kalamas, T., Stefanidou, M., Bikiaris, N. D., Vlachopoulos, A., Koumentakou, I., & Bikiaris, D. N. (2021). Poly(lactic Acid): A Versatile Biobased Polymer for the Future with Multifunctional Properties—From Monomer Synthesis, Polymerization Techniques and Molecular Weight Increase to PLA Applications. Polymers, 13(11), 1822. https://doi.org/10.3390/polym13111822
Sutiono, S., Teshima, M., Beer, B., Schenk, G., & Sieber, V. (2020). Enabling the Direct Enzymatic Dehydration of d-Glycerate to Pyruvate as the Key Step in Synthetic Enzyme Cascades Used in the Cell-Free Production of Fine Chemicals. ACS Catalysis, 10(5), 3110-3118. https://doi.org/10.1021/acscatal.9b05068
Prestes, P. S., de Oliveira, M. M. M., & Leonardi, G. R. (2013). Randomized clinical efficacy of superficial peeling with 85% lactic acid versus 70% glycolic acid. Anais brasileiros de dermatologia, 88(6), 900-905. https://doi.org/10.1590/abd1806-4841.20131888
O'Connor, A. A., Lowe, P. M., Shumack, S., & Lim, A. C. (2018). Chemical peels: A review of current practice. Australasian Journal of Dermatology, 59(3), 171-181. https://doi.org/10.1111/ajd.12715
Ghersetich, I., Brazzini, B., Peris, K., Cotellessa, C., Manunta, T. & Lotti, T. (2004). Pyruvic Acid Peels for the Treatment of Photoaging. Dermatologic Surgery, 30(1), 32-36. https://doi.org/10.1111/j.1524-4725.2004.30002.x
Apotheke des Universitátsklinikums Heidelberg (17 Jul. 2015). Pflegesalbe 100 g. Obtainable at: https://www.klinikum.uni-heidelberg.de/fileadmin/apotheke/Rezeptur-Vorschriften/Pflegesalbe.pdf (accessed on 24.10.2022)
Tan, H. W., Abdul Aziz, A. R., & Aroua, M. K. (2013). Glycerol production and its applications as a raw material: A review. Renewable and Sustainable Energy Reviews, 27, 118-127. https://doi.org/10.1016/j.rser.2013.06.035
Rodrigues, A., Bordado, J. C., & dos Santos, R. G. (2017). Upgrading the Glycerol from Biodiesel Production as a Source of Energy Carriers and Chemicals—A Technological Review for Three Chemical Pathways. Energies, 10(11), 1817. https://doi.org/10.3390/en10111817
Wang, Z. X., Zhuge, J., Fang, H., & Prior, B. A. (2001). Glycerol production by microbial fermentation: a review. Biotechnology Advances, 19(3), 201-223. https://doi.org/10.1016/s0734-9750(01)00060-x
Aslankoohi, E., Rezaei, M. N., Vervoort, Y., Courtin, C. M., & Verstrepen, K. J. (2015). Glycerol Production by Fermenting Yeast Cells Is Essential for Optimal Bread Dough Fermentation. PLOS ONE, 10(3), e0119364. https://doi.org/10.1371/journal.pone.0119364
IRENA & METHANOL INSTITUTE (2021). Innovation Outlook: Renewable Methanol, International Renewable Energy Agency, Abu Dhabi. https://www.irena.org/publications/2021/Jan/Innovation-Outlook-Renewable-Methanol (accessed on 19.10.2022)
Liepins, J., Kuorelahti, S., Penttila, M., & Richard, P. (2006). Enzymes for the NADPH-dependent reduction of dihydroxyacetone and D-glyceraldehyde and L-glyceraldehyde in the mould Hypocrea jecorina. The FEBS Journal, 273, 4229-4235. https://doi.org/10.1111/j.1742-4658.2006.05423.x
Wang, J., Qu, G., Xie, L., Gao, C., Jiang, Y., Zhang, Y.-H. P. J., Sun, Z., & You, C. (2022). Engineering of a thermophilic dihydroxy-acid dehydratase toward glycerate dehydration for in vitro biosystems. Applied Microbiology and Biotechnology, 106(9-10), 3625-3637. https://doi.org/10.1007/s00253-022-11936-9
Hromádková, Z., & Ebringerová, A. (1995). Isolation and Characterization of Hemicelluloses of Corn Hulls. Chemical Papers, 49(2), 97-101. https://chempap.org/?id=7&paper=3556
Collins, S. R. A., Wellner, N., Martinez Bordonado, I., Harper, A. L., Miller, C. N., Bancroft, I., & Waldron, K. W. (2014). Variation in the chemical composition of wheat straw: the role of tissue ratio and composition. Biotechnology for Biofuels, 7, 121. https://doi.org/10.1186/s13068-014-0121-y
Kornecki, J. F., Carballares, D., Tardioli, P. W., Rodrigues, R. C., Berenguer-Murcia, A., Alcantara, A. R., & Fernandez-Lafuente, R. (2020). Enzyme production of D-gluconic acid and glucose oxidase: successful tales of cascade reactions. Catalysis Science & Technology, 10(17), 5740-5771. https://doi.org/10.1039/DOCY00819B
Entner, N., & Doudoroff, M. (1952). GLUCOSE AND GLUCONIC ACID OXIDATION OF PSEUDOMONAS SACCHAROPHILA. Journal of Biological Chemistry, 196(2), 853-862. https://doi.org/10.1016/S0021-9258(19)52415-2
Conway, T. (1992). The Entner-Doudoroff pathway: history, physiology and molecular biology. FEMS Microbiology Reviews, 9(1), 1-27. https://doi.org/10.1111/j.1574-6968.1992.tb05822.x
Peekhaus, N., & Conway, T. (1998). What's for Dinner?: Entner-Doudoroff Metabolism in Escherichia coli. Journal of Bacteriology, 180(14), 3495-3502. https://doi.org/10.1128/JB.180.14.3495-3502.1998
Reher, M., Fuhrer, T., Bott, M., & Schönheit, P. (2010). The Nonphosphorylative Entner-Doudoroff Pathway in the Thermoacidophilic Euryarchaeon Picrophilus torridus Involves a Novel 2-Keto-3-Deoxygluconate-Specific Aldolase. Journal of Bacteriology, 192(4), 964-974. https://doi.org/10.1128/JB.01281-09
Sutter, J. M., Tastensen, J. B., Johnsen, U., Soppa, J., & Schönheit, P. (2016). Key Enzymes of the Semiphosphorylative Entner-Doudoroff Pathway in the Haloarchaeon Haloferax volcanii: Characterization of Glucose Dehydrogenase, Gluconate Dehydratase, and 2-Keto-3-Deoxy-6-Phosphogluconate Aldolase. Journal of Bacteriology, 198(16), 2251-2262. https://doi.org/10.1128/JB.00286-16
Lamble, H. J., Heyer, N. I., Bull, S. D., Hough, D. W., & Danson, M. J. (2003). Metabolic Pathway Promiscuity in the Archaeon Sulfolobus solfataricus Revealed by Studies on Glucose Dehydrogenase and 2-Keto-3-deoxygluconate Aldolase. Journal of Biological Chemistry, 278(36), 34066-34072. https://doi.org/10.1074/jbc.M305818200
Theodossis, A., Walden, H., Westwick, E. J., Connaris, H., Lamble, H. J., Hough, D. W., Danson, M. J., & Taylor, G. L. (2004). The Structural Basis for Substrate Promiscuity in 2-Keto-3-deoxygluconate Aldolase from the Entner-Doudoroff Pathway in Sulfolobussol fataricus. Journal of Biological Chemistry, 279(42), 43886-43892. https://doi.org/10.1074/jbc.M407702200
Lin, B., & Tao, Y. (2017). Whole-cell biocatalysts by design. Microbial Cell Factories, 16, 106. https://doi.org/10.1186/s12934-017-0724-7
Moore, S. & Link, K. P. (1940). CARBOHYDRATE CHARACTERIZATION: I. THE OXIDATION OF ALDOSES BY HYPOIODITE IN METHANOL II. THE IDENTIFICATION OF SEVEN ALDO-MONOSACCHARIDES AS BENZIMIDAZOLE DERIVATIVES. Journal of Biological Chemistry, 133, 293-311. https://doi.org/10.1016/S0021-9258(18)73312-7
Setubal, J. C., dos Santos, P., Goldman, B. S., Ertesv\dot{a}g, H., Espin, G., Rubio, L. M., Valla, S., Almeida, N. F., Balasubramanian, D., Cromes, L., Curatti, L., Du, Z., Godsy, E., Goodner, B., Hellner-Burris, K., Hernandez, J. A., Houmiel, K., Imperial, J., Kennedy, C., Larson, T. J., Latreille, P., Ligon, L. S., Lu, J., Mark, M., Miller, N. M., Norton, S., O'Carroll, I. P., Paulsen, I., Raulfs, E. C., Roemer, R., Rosser, J., Segura, D., Slater, S., Stricklin, S. L., Studholme, D. J., Sun, J., Viana, C. J., Wallin, E., Wang, B., Wheeler, C., Zhu, H., Dean, D. R., Dixon, R., & Wood, D. (2009). Genome sequence of Azotobacter vinelandii, an obligate aerobe specialized to support diverse anaerobic metabolic processes. Journal of Bacteriology, 191(14), 4534-4545. https://doi.org/10.1128/JB.00504-09
Protein [Internet]. Bethesda (MD): National Library of Medicine (US), National Center for Biotechnology Information. Accession No. AC081022.1, L-arabonate dehydratase [Azotobacter vinelandii DJ]. Obtainable at: https://www.ncbi.nlm.nih.gov/protein/226721851 (accessed on 04.10.2022)
Wolterink-van Loo, S., Siemerink, M. A. J., Perrakis, G., Kaper, T., Kengen, S. W. M., & van der Oost, J. (2009). Improving low-temperature activity of Sulfolobus acidocaldarius 2-keto-3-deoxygluconate aldolase. Archaea, 2(4), 233-239. https://doi.org/10.1155/2009/194186
de Faria, J. T., Sampaio, F. C., Converti, A., Lopes Passos, F. M., Minim, V. P., & Minim, L. A. (2009). Use of response surface methodology to evaluate the extraction of Debaryomyces hansenii xylose reductase by aqueous two-phase system. Journal of Chromatography B: Analytical Technologies in the Biomedical and Life Sciences, 877(27), 3031-3037. https://doi.org/10.1016/j.jchromb.2009.07.023
Zheng, Y., Guo, S., Guo, Z., & Wang, X. (2004). Effects of N-terminal deletion mutation on rabbit muscle lactate dehydrogenase. Biochemistry (Moscow), 69(4), 401-406. https://doi.org/10.1023/b:biry.0000026195.31821.e1
Lamed, R., & Zeikus, J. G. (1980). Ethanol production by thermophilic bacteria: relationship between fermentation product yields of and catabolic enzyme activities in Clostridium thermocellum and Thermoanaerobium brockii. Journal of Bacteriology, 144(2), 569-578. https://doi.org/10.1128/jb.144.2.569-578.1980
Chinnawirotpisan, P., Matsushita, K., Toyama, H., Adachi, O., Limtong, S., & Theeragool, G. (2003). Purification and characterization of two NAD-dependent alcohol dehydrogenases (ADHs) induced in the quinoprotein ADH-deficient mutant of Acetobacter pasteurianus SKU1108. Bioscience, Biotechnology, and Biochemistry, 67(5), 958-965. https://doi.org/10.1271/bbb.67.958
Protein [Internet]. Bethesda (MD): National Library of Medicine (US), National Center for Biotechnology Information. Accession No. WP_013168047.1, NAD-dependent formate dehydrogenase [Starkeya novella]. Obtainable at: https://www.ncbi.nlm.nih.gov/protein/WP_013168047.1 (accessed on 30.10.2023)

1. A method for preparing an aqueous solution containing glycerol and a salt of pyruvic acid or lactic acid by treating a salt of gluconic acid and/or galactonic acid in an aqueous alcohol-containing solution in vitro with an enzyme system comprising a dehydratase, an aldolase, an alcohol dehydrogenase together with a cofactor, a glyceraldehyde reductase together with a cofactor and, optionally, a lactate dehydrogenase together with a cofactor, whereby an aqueous solution containing glycerol and a salt of pyruvic acid or lactic acid is obtained, from which the enzyme system is separated and which is optionally concentrated.

2. The method according to claim 1, wherein the salt of gluconic acid and/or of galactonic acid is an alkali salt and is present in the alcohol-containing solution in a concentration of between 50 and 350 g/l.

3. The method according to claim 2, wherein the alkali salt is present in a concentration of between 150 and 300 g/l.

4. The method according to claim 3, wherein the alkali salt is present in a concentration of between 150 and 250 g/l.

5. The method according to claim 1, wherein the alcohol in the alcohol-containing solution is isopropanol.

6. The method according to claim 1, wherein the enzymes forming the enzyme system are present as a lysate of the corresponding cells forming them.

7. The method according to claim 1, wherein the enzymes forming the enzyme system are present as a homogenate of the corresponding cells forming them.

8. The method according to claim 1, wherein the enzymes forming the enzyme system are present as a suspension of the corresponding cells forming them.

9. The method according to claim 1, wherein the treatment with the enzyme system is carried out as a one-pot process.

10. An aqueous solution comprising glycerol and a salt of pyruvic acid or lactic acid obtainable by a method according to claim 1.
